(19) United States
(12) Patent Application Publication
(10) Pub. No.: US 2024/0181169 A1
(43) Pub. Date: Jun. 6, 2024

(54) CAPPED SYRINGES AND METHODS OF SEALING SAME

(71) Applicant: DiscGenics, Inc., Salt Lake City, UT (US)

(72) Inventors: Joel Salem, Salt Lake City, UT (US); Isaac Erickson, Layton, UT (US); Anthony Mangum, Centerville, UT (US); Ryan Radina, Salt Lake City, UT (US); Haeli Hauritz, Park City, UT (US)

(21) Appl. No.: 18/526,510

(22) Filed: Dec. 1, 2023

Related U.S. Application Data
(60) Provisional application No. 63/429,439, filed on Dec. 1, 2022.

(51) Int. Cl. A61M 5/32; A61M 5/31

(52) U.S. Cl. CPC A61M 5/3202; A61M 5/3134

(57) ABSTRACT

A syringe having one or more structures or features for maintaining integrity of separation from outside environment is disclosed, the syringe may have a threaded barrel, or a barrel cap, or a needle tip cap configured to allow for inductive sealing against ingress of at least one contaminant. In some embodiments, the barrel cap may include one or more structures for securing the cap to the barrel end by applying a screwing force, a twisting force and/or a snap/press force to the cap.

### CROSS-REFERENCE TO RELATED APPLICATIONS

This application claims priority to U.S. Provisional Application No. 63/429,439 filed Dec. 1, 2022, entitled “CAPPED SYRINGES AND METHODS OF SEALING SAME,” which is incorporated by reference herein, in its entirety and for all purposes.

### FIELD

The disclosed processes, methods, and systems are directed to devices, systems, and methods for preserving therapeutic mammalian cells in storage vessels at low temperatures without ingress of gasses or liquids, wherein the vessels are suitable for administering the therapeutic mammalian cells to a subject in need thereof.

### BACKGROUND

Therapeutic mammalian cells require very cold temperatures for storage, maintenance, and transportation. Prior to administration to a subject, these cells must be thawed and placed in a delivery device. These very low temperatures are necessary for preserving and protecting their therapeutic viability and efficacy. However, the environments necessary to achieve these very low temperatures may also result in failures of the storage vessels, leading to contamination.

Traditional cryo-preservation methods often result in inconsistent or poor container closure below −135° ° C. In addition, traditional methods of cryopreservation rely on additional manipulation steps to transfer the cells to a device capable of administering them to the subject. Moreover, these steps increase the risk of the cells being contaminated by unwanted bacteria, viruses, chemicals, etc.

What is needed are improved devices, systems and methods for freezing, storing, maintaining, transporting, and administering therapeutic mammalian cells.

### SUMMARY

Disclosed herein are devices, systems, and methods that improve the freezing, storing, maintaining, transporting, and administering of therapeutic cells with reduced manipulation and risks for contamination. In one example, a barrel cap for scaling a syringe barrel is disclosed, wherein the barrel cap may include an exterior surface and an interior surface including one or more structures for removably engaging one or more complementary structures at or near a barrel end of the syringe. In some examples, the one or more structures may include a plurality of tabs spaced about the interior surface, and/or the one or more structures may engage a side of the complementary structures extending away from the barrel end. In some examples, the one or more structures may include cam structures, snap tabs, and/or a first threading, for example wherein the complementary structures include a second threading. In some examples, the exterior surface may define ribs.

Also disclosed are needle caps, that may be useful for scaling a syringe tip. The needle cap may include a user engagement surface and a syringe needle end engagement surface, wherein the needle engagement surface may define threaded structures for engaging complementary threading structures at an interior wall of a needle hub lumen. In some examples, the needle cap may include an elastomeric stopper for engaging a needle tip orifice defined by the needle hub lumen. In some examples, the needle engagement surface is spaced inwardly from the user engagement surface.

Also disclosed are syringe devices, for example the syringe device may include a barrel having an interior and an exterior surface, the interior surface may at least partially define an interior cavity for holding a therapeutic composition, a barrel end, wherein a flange may be positioned at or near the barrel end, and a needle end. In some examples, the flange may extend outward asymmetrically about the barrel orifice. In some examples, the syringe device may include includes a barrel cap removably engaged to the barrel end. In some examples, the barrel cap may removably engage the flange and/or the syringe device may include threading structures positioned about the barrel orifice that may be useful for engaging a threaded barrel cap. In some of these examples, the threading structures about the barrel orifice may be located above the flange. In some examples, the syringe device may include therapeutic mammalian cells within the interior cavity, wherein the therapeutic mammalian cells may be a component of the therapeutic composition.

Also disclosed are methods for preventing ingress of one or more contaminants into a syringe lumen, the method including sealing the syringe lumen at a first, barrel end and/or a second needle end. In some examples, the method may include aseptically filling an interior cavity of the syringe, induction sealing a cap over an orifice of the syringe, and/or freezing the syringe.

Also disclosed are methods of inductively sealing a syringe the method including placing an inductive seal at or near a barrel end opening, applying a compressive force to the inductive seal, in the direction of the barrel end opening, and subjecting the inductive seal to an electromagnetic field, wherein the electromagnetic field may be sufficient to heat a metal portion of the inductive seal to a temperature sufficient to melt a polymer of the inductive seal. In some examples, the method may include loading the syringe lumen with a therapeutic mammalian cell composition before placing an inductive seal.

### BRIEF DESCRIPTION OF THE DRAWINGS

FIG. 1 is a schematic diagram of one embodiment of the disclosed barrel syringe and needle tip cap.

FIG. 2A shows a second embodiment of the disclosed barrel syringe, a barrel cap, and a needle tip cap.

FIG. 2B shows one embodiment of the disclosed barrel cap for twist application including an induction seal.

FIG. 3A shows a perspective view of one embodiment of the disclosed barrel cap for snap or twist application.

FIG. 3B shows a plan view of one embodiment of the disclosed barrel cap for snap or twist application.

FIG. 3C shows a cross sectional view of the barrel cap of FIG. 3B taken along line 3C-3C.

FIG. 3D shows one embodiment of the disclosed barrel cap and an embodiment of a barrel syringe the barrel cap may seal.

FIG. 4A shows a perspective view of one embodiment of the disclosed barrel cap for twist, snap, and/or press application.

FIG. 4B shows a plan view of one embodiment of the disclosed barrel cap for snap or press application.

FIG. 4C shows a cross sectional view of the disclosed barrel cap of FIG. 4B taken along line 4C-4C.

FIG. 4D shows one embodiment of the disclosed barrel cap and an embodiment of a barrel syringe the barrel cap may seal. Note the barrel syringe is not completely depicted.

FIG. 5A shows one embodiment of the disclosed needle tip cap with a gasket, or O-ring, and a tip orifice elastomeric stopper.

FIG. 5B shows one embodiment of the disclosed needle tip cap and the gasket or O-ring.

FIG. 5C shows a perspective view of the needle tip cap of FIG. 5B.

FIG. 6 is a flow diagram depicting one embodiment of a disclosed method.

### DETAILED DESCRIPTION

Disclosed herein are devices, systems, and methods for preservation and handling of therapeutic mammalian cells. In some embodiments, the disclosed device may include a cap, a storage vessel, and/or a syringe barrel providing enhanced preservation, handling, and administration of therapeutic mammalian cells. In some embodiments, the disclosed device may be configured to interact with existing devices to create a system with enhanced characteristics of preservation and handling of therapeutic mammalian cells. In many embodiments, methods of using the disclosed devices and or systems for enhancing preservation and handling of therapeutic mammalian cells, relative to existing methods, is disclosed.

The disclosed devices, systems, and methods may be configured to provide secondary and/or primary seals to preservation devices. The disclosed devices, systems, and methods may be configured to provide enhanced sealing at ultra-cold (ultra-low) or cryo-temperatures. Ultra-cold temperatures may be temperatures below about −70° C. Cryo-temperatures may be temperatures below about −120° C., −130° C., 140° C., −150° C., −160° C., −170° C., −180° C., or −190° C.

The disclosed devices, systems, and methods are useful in sealing syringes of various materials. In many embodiments, the materials used to manufacture the disclosed syringe bodies may resist excessive expansion, contraction, breakage, cracking, shattering, etc. when frozen and thawed. In one embodiment, the syringe body may comprise a polymeric glass substitute, for example a polymeric cyclic olefin such as crystal zenith, or cyclo olefin polymer and copolymers.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The disclosed devices and systems may be useful as a pre-filled syringe (PFS). For example, syringes for storing, freezing, maintaining, and administering therapeutic mammalian cells. Typically, PFSs are not intended for use with therapeutic mammalian cells, and lack sufficient structures or systems to preserve sterility at temperatures necessary for mammalian cells—such as secondary seals—and are therefor ineffective at maintaining a sterile closed container at ultra-cold temperature. Existing PFSs also lack primary and secondary seals capable of providing sufficient sealing integrity at ultra-cold and cryo-temperatures, and/or within environments necessary to maintain such temperatures. For example, existing PFS systems are constructed of materials that are not compatible and/or possess significantly different performance characteristics (contracting, expanding, resilience, etc.) at temperatures and environments necessary for maintaining therapeutic mammalian cells. Thus, existing PFS and PFS systems result in container closure integrity failures at these temperatures.

Disclosed herein, are various secondary sealing devices, mechanisms, and systems useful in ensuring and maintaining storage vessel closure integrity at, or below, ultra-cold temperatures and environments for maintaining same. In many embodiments, storage vessel closure integrity may be assessed by monitoring ingress and/or egress of nitrogen from the interior of the storage vessel to the exterior environment. In some cases, this may be performed by analyzing gasses within the lumen of the vessel after freezing and thawing.

### Syringe and Flange Design

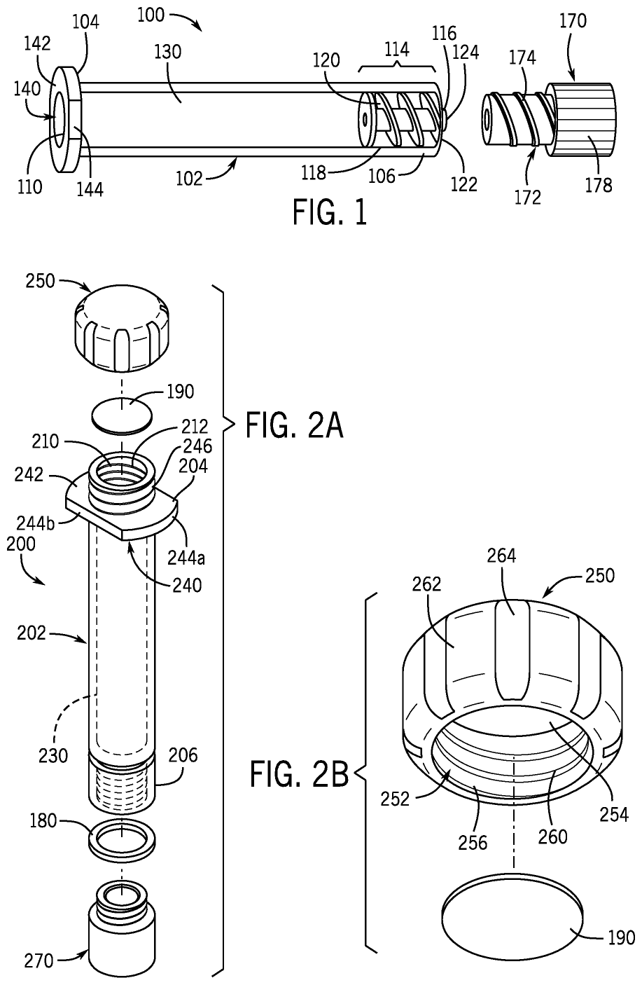

With reference to the Figures, various storage vessels 100 may be configured to receive the various caps, such as a barrel cap 250, or needle end caps 170, 270. In many embodiments, the vessel 100 may store therapeutic mammalian cells. In some embodiments, the vessel 100 may be a syringe, one embodiment of which is shown in FIG. 1. Additional embodiments 200, 300, 400 are shown in FIGS. 2A, 3D, and 4D, respectively. In one embodiment, the syringe 100 may be a pre-filled syringe. The syringe 100 may include a barrel 102 having a first plunger end 104 and second needle end 106, at the opposite end from the plunger end 104. In many embodiments, the barrel 102 may be substantially cylindrical, defining a hollow space 130 within the barrel 102 that may be configured to receive the therapeutic mammalian cells.

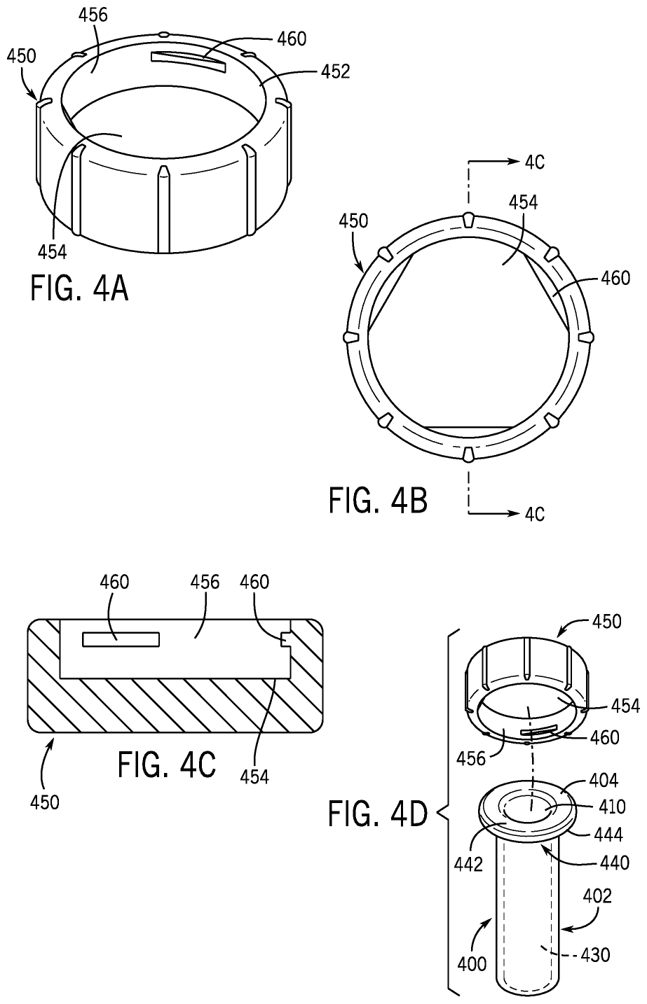

The plunger end 104 may define a barrel orifice 110 in fluid connection with the hollow space 130. The syringe 100 may define complementary structures 140 about the barrel orifice 110 to receive a scaling cap 250 (shown in FIGS. 2A and 2B). The complementary structures 140 may include a flange 142, threading 246, or similar structures. The flange 142 may be positioned about the barrel orifice 110 defining an opening extending to a barrel lumen 130 which may be configured to contain therapeutic mammalian cells, and may be sealed by a barrel sealing cap, which may be similar to barrel cap 450 of FIGS. 4A-4D. In the embodiment of FIG. 1, the needle end 106 may include a tip 116 and a needle hub lumen 122 positioned about the tip 116. The tip 116 may be configured to accept a syringe needle (not shown) and/or a needle tip cap structure 170.

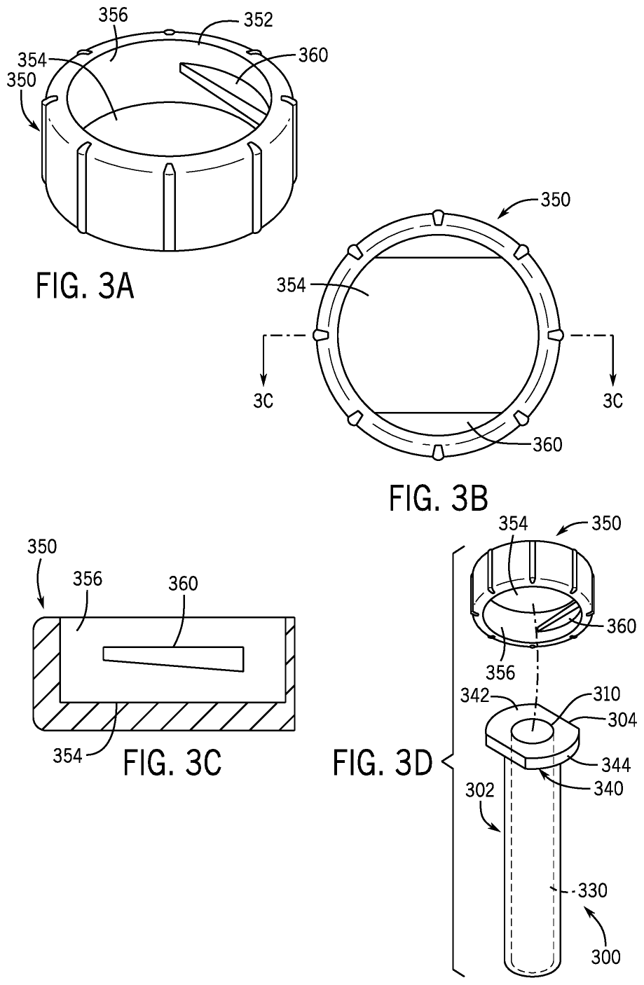

The flange 142 at the plunger end 104 may generally extend outward from the barrel orifice 110. The flange 142 be any of a variety of shapes. For example, as shown in FIGS. 1 and 4D the flange 142, 442 may extend generally annularly around the barrel orifice 110, 410 and away from the barrel 102, 402. For example, when the flange 442 is symmetrical or near symmetrical around the barrel orifice 410, such as the configuration in FIG. 4D, the flange 442 may be referred to as a circular flange 442. The flange 142, 242, 342 may have edges 144, 244, 344 spaced at a lesser distance from the barrel orifice 210, 310 and barrel as compared to the rest of the flange 242, 342, as shown in FIGS. 2A and 3D. The example shown in FIGS. 2A and 3D may be referred to as a Double-D configuration 242, 342. The underside of a scaling cap, such as scaling caps 350, 450, or a feature 360, 460 of the sealing caps 350, 450, may be placed over the flange 342, 442 around the edges 344, 444, and in some examples then moved, such that the features 344, 444 engage the flange 342, 444 from an underside of the flange 342, 444.

In one embodiment, the disclosed syringe barrels 202, as shown in FIG. 2A, may include an additional threaded surface 246 positioned at or near the barrel end 204. The threaded surface 246 may extend axially from the flange 242. For example, the flange 242 may be below the threaded surface 246 as depicted in FIG. 2. In other examples, the threading 246 may be defined by the interior 212 of the barrel orifice 210. In these embodiments, the sealing cap 250 may define complimentary features 260 and the threading 246 may aid in sealing the barrel end 204. In some examples, a sealing structure 180, 190 may be positioned between the barrel end 204 and the cap floor 254—with the threading 246 aiding in applying the necessary force to install the sealing structure 180, 190.

The needle end 106 may define a needle hub 114. The needle hub 114 may define threading 120 on the interior of the syringe 100 configured to receive a tip cap 170. For example, the threading 120 may be defined by an inner wall 118 of the needle hub 114. The needle hub 114 may further include a tip 116, where the tip 116 may be an extension from the needle hub 114 and configured to receive a needle (not shown).

In some examples, a plunger may be inserted into the interior volume or lumen 130 at the plunger end 104, such as at the barrel orifice 110. The plunger may be configured to be pushed through the lumen toward the needle end expelling the contents of the syringe lumen, for example a therapeutic composition. In some embodiments, the plunger may have at least one structure for accepting a plunger rod, that may be useful in pushing the plunger through the lumen. The plunger may include one, two or more separable components. In some embodiments, a plunger component may be configured to at least partially seal the orifice 110, such as a stopper or gasket. In some examples, the plunger, or a portion of the plunger, may be inserted into the lumen 130, 230 prior to placing a sealing cap 250 on the barrel end 104, 204.

### Sealing Caps

Disclosed herein are various structures configured to seal a storage vessel 100. For example, scaling caps 250, 170. With reference to FIGS. 2A and 2B, the disclosed cap 250, may be configured to seal a syringe barrel 202 at the plunger end 204 (a barrel cap 250, as shown in FIG. 2B) or a needle-end cap 270 (see FIG. 2A). In many embodiments, with reference to FIGS. 3A to 4D, the disclosed barrel cap 250, 350, 450 is configured to engage the syringe barrel flange 342, 442 at or near the plunger end 304, 404 of the syringe barrel 202. Because the barrel flange 242, 342, 442 may be of various shapes the cap 250, 350, 450 may be configured to engage the variety of flange shapes, such as a circular barrel flange 142, 442 or a Double D barrel flange 242, 342 (e.g. a flange 242 having to rounded edges 244a, 344a and two straight edges 244b, 344b; as shown in FIGS. 2A and 3D). The barrel caps 250, 350, 450 may be configured to engage the flange 242, 342, 442, by press fit, snap fit, or twisting. In many embodiments, the cap walls 256, 356, 456 may include one or more structures for engaging the barrel flange 242, 342, 442, such as a tab 442, threading 242, or a cam 342. For example, two or more tabs 442 or cams 342.

The sealing structure 250, 170 may again include one or more of a seal 190, gasket 180, or the like. In some embodiments, the sealing structure 250, 170 may include an induction seal 190 and/or a gasket 180. For example, an induction seal 190 may be pre-installed within the cap 250, 170.

### Twist Cap and Threaded Cap

A barrel cap 250, 350 configured to seal by a twisting action may be a twist cap (see a discussion of twist caps below) or a threaded cap 250, 350. Where engagement is via threading/screwing of the barrel cap 250, the threaded cap may include threading 260 on the interior 252 of the cap 250, as shown in FIG. 2A. In these embodiments, the barrel cap 250 may include the complementary threading 260 at the cap wall 256 for engaging the threading 220 on the barrel end 204. The barrel end 204 may define complementary structures 240 to engage with the barrel cap 250, such as flanges 242 or threading 246. The threading 260 on the interior 252 of the threaded cap 250 may align with, and receive, the threading 246 on the exterior of the plunger end 204 of the syringe 200. As the threaded cap 250 is rotated, the threaded cap 250 may be pressed against the barrel orifice 210. The threaded cap 250 may further include ribs 264 around the exterior 262 of the threaded cap 250 to assist a user in applying the twisting force to the threaded cap 250. The interior surface 212 of the barrel end 204, opposite the exterior surface where threads 246 are located, may be smooth to allow for the insertion of a stopper feature.

Where engagement is via a twisting of the barrel cap 350, the twist cap 350 may include one or more cam structures 360 extending from the cap wall 356 within the interior of the barrel cap 352 (see FIGS. 3A-3D). Twisting the twist cap 350 may aid in sealing the barrel end 304 of the syringe 300, by positioning the cap floor 354 proximal the barrel end 304, and exerting pressure against the barrel end 304. The barrel end 304 may define complementary structures 340 to engage with the barrel cap 350, such as flanges 342. In these embodiments, twisting may aid in applying the sealing structure 180, 190 to the barrel end 204, 304 and covering the barrel orifice 210, 310. In some embodiments, the barrel 204, 304 may be sealed with an elastomeric ring (not shown, but similar to 180) that may be positioned before the barrel cap 250, 350 is positioned at the barrel end 204, 304.

The twist cap 350 may be configured for a double-D type flange 242, 342. With a double-D type flange 342, the cam structures 360 may slide around the edges 344 of the Double-D flange 342 until the barrel orifice 310 is in contact with the cap floor 354. The twist cap 350 may then rotate to slide the cam structures 360 under the flange 342. The cam structures 360 may be angled such that a greater sealing force is induced as the twist cap 360 rotates relative to the flange 342.

In various embodiments, the barrel cap 250, 350 may include one or more sealing structures 180, 190 within the interior of the cap. The sealing structures 180, 190 may be added to the cap interior 252, 352, at or near the cap floor 254, 354, or the sealing structures 180, 190 may be pre-installed. The sealing structures 180, 190 may be separate structures such as a gasket 180 or an induction seal 190, as shown in FIG. 2A or 2B.

An induction seal 190 may be placed between the barrel cap 250 and a barrel orifice 210. An induction seal 190 may generate heat when exposed to an electrical current and cause a portion of the seal 190 to melt and bond with the syringe 200 around the barrel orifice 210. An induction seal 190 may include a sealing layer and a conductive layer. The conductive layer may include a conductive material, such as aluminum, and generate heat when subjected to an electrical power source. The seal layer may be a material that melts at the temperature the conductive material reaches, such as a polymer film.

The gasket or sealing structure 180 may be a variety of shapes. In some embodiments, the sealing structure 180 may be a gasket having a rectangular cross section, an O-ring having a circular cross section, or a washer having an elongated or thin cross section. In some examples, the gasket 180 may be included on the plunger end 204 of the barrel 202 and/or the needle end 106. The gasket 180 may be an annular structure that aligns around the barrel orifice 210 or with a portion of the barrel cap 250. The gasket 180 may be an elastomer, for example a polymeric elastomer. In some embodiments, the gasket 180 may be manufactured from bromobutyl or a bromobutyl formulation. When the cap 250 is twisted, the cap floor 254 may contact the gasket 180 and exert a pressure exerted, the gasket 180 may deform under the pressure to create a seal between the cap 250 and barrel 202 to aid in sealing the barrel orifice 210.

### Snap Caps

Where engagement of the barrel cap 450 is via press- or snap-fitting, the cap wall 456 may include two or more tabs 460 extending from the cap wall 456 inward toward the center of the cap 450. The tabs 460 may be snap tabs. In these embodiments, as with the twist caps 250, 350, discussed herein, scaling structures 180 may be positioned within the cap interior 452 proximal to the cap floor 454, as shown in FIGS. 4A-4D. The barrel end 404 may define complementary structures 440 to engage with the barrel cap 450, such as flanges 442. In many embodiments, the snap cap 450 includes at least one pre-installed induction seal 190 designed to engage the barrel end 404 when the cap 450 is snapped onto the flange 442 of an open barrel of a plastic parenteral grade syringe 400. In many embodiments, the induction seal 190 is held in place against the barrel end 404 while the seal 190 is subjected to an electromagnetic field.

Snap caps 450 may be useful where the barrel flange 442 is round, such as with the circular flange type 142, 442. In these embodiments, the cap wall 456 may include three or more tab structures 460 for engaging the flange 442. As discussed with reference to the twist cap 250 above, the syringe 400 may include a stopper within the lumen 410 of the barrel 402 prior to snapping the cap 450 in place.

### Syringe Tip Cap

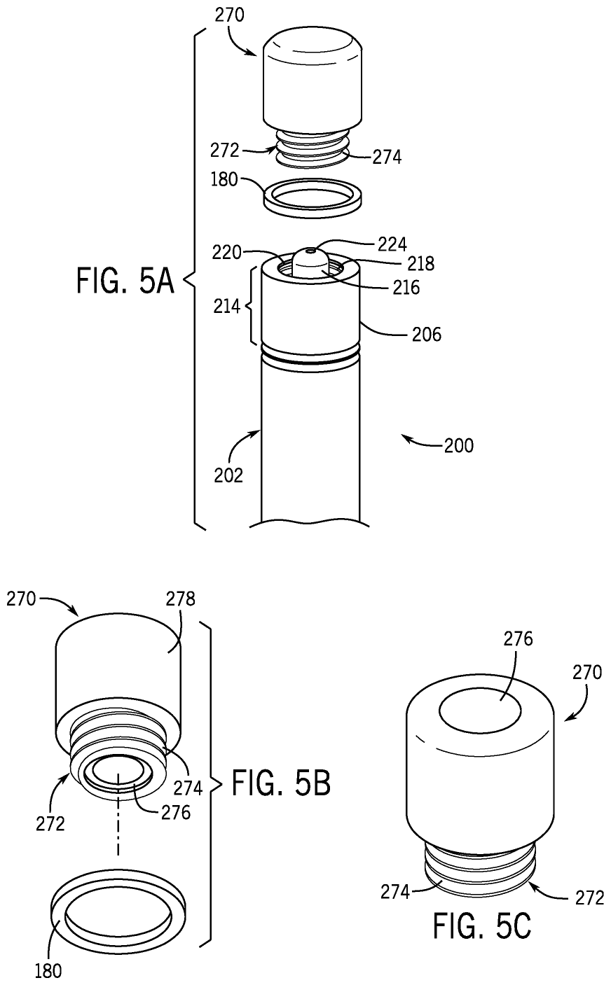

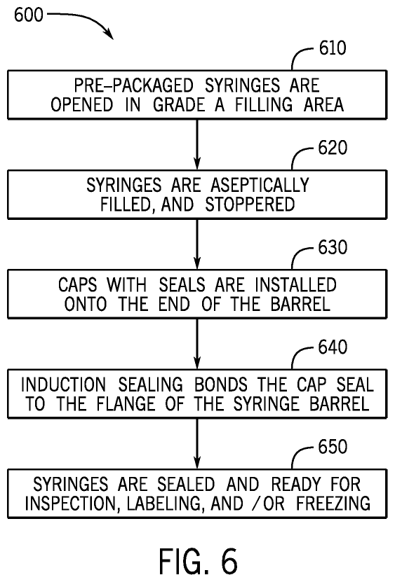

Examples of a threaded syringe needle tip cap 170, 270 are shown in FIGS. 1, 2A and 5A to 5B. The tip cap 170, 270 may define a needle end engagement surface 172, 272 including threading 174, 274. In some examples, threading may be positioned within an interior cavity of the cap. The threading 174, 274 may engage with the threading 120, 220 or inner wall 118, 218 of the needle tip end 106, 206. In these embodiments, the tip cap 170, 270 may include an elastomeric stopper or cap 276 for inserting into the orifice 124, 224 of the syringe tip 106, 206. In some embodiments the elastomeric stopper 276 may be affixed to the cap 270, or may be a separate structure installed prior to affixing the needle tip cap 270. In many cases, the cap 270 may encapsulate the elastomeric stopper 276, backing and compressing it while it is twisted on, ensuring a tight fit with the open tip 106, 206. This may ensure a long-term seal, for example by allowing the elastomeric 276 and plastic portions 106, 206 to adhere over time, for example, between the manufacturing and use. In various embodiments, the syringe tip cap 170, 270 may include an elastomeric gasket or ring 180, for example an O-ring, as shown in FIG. 5. The syringe needle tip cap 170, 270 may include an outer surface 178, 278, or user engagement surface, that provides grip for a user to twist or control the needle tip cap 170, 270, as may be shown in FIG. 1A.

### Methods

Various methods and processes are disclosed for installing scaling caps 170, 270, 250, 350, 450 on syringe barrels 102, 202, 302, 402. In one embodiment, the disclosed methods may include opening syringes 100, 200, 300, 400 in a clean room area; filing the syringes 100, 200, 300, 400 aseptically by placing a therapeutic mammalian cell composition within the lumen or volume 130, 230, 330, 430 of the syringe barrel 102, 202, 302, 402; placing a stopper within the lumen or volume 130, 230, 330, 430 of the syringe barrel 102, 202, 302, 402, proximal the composition; scaling the barrel end orifice 110, 210, 310, 410 with a seal structure 180, 190, using, for example induction sealing. In many embodiments, a needle tip cap 170, 270 may be positioned at the needle end 106, 206 and a sealing structure 180, 190 positioned between the needle tip cap 170, 270 and the tip orifice 124, 224, prior to filing the syringe barrel 102, 202, 302, 402. In many embodiments, the sealing structure 180, 190 may be induction sealed to the syringe barrel 102, 202, 302, 402.

In one example of a method 600 is disclosed. The method 600 may include step 610 where pre-packaged syringes 100, 200, 300, 400 are opened in a Grade A filling area. The method 600 may include step 620 where the syringes 100, 200, 300, 400 are aseptically filled, and stoppered. The method 600 may include step 630 where caps 170, 270, 250, 350, 450 with seals are installed onto the end 104, 204, 304, 404, 106, 206, 306, 406 of the barrel 102, 202, 302, 402. The method 600 may include step 640 where induction scaling bonds the cap seal 190 to the flange 142, 242, 342, 442 of the syringe barrel 102, 202, 302, 402. The method 600 may include step 650 where the syringes 100, 200, 300, 400 are ready for inspection, labeling, and/or freezing.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims. For example, it is appreciated that at least some features described with respect to one embodiment may be interchangeably included with or replace features described with respect to a second embodiment.

We claim:

1. A syringe device, comprising:
a barrel, having an interior and an exterior surface, the interior surface at least partially defining an interior cavity for holding a therapeutic composition;
a barrel end, wherein a flange is positioned at or near the barrel end;
a barrel cap configured to attach to the barrel end; and
a needle end.

2. The syringe device of claim 1, wherein the barrel end defines a barrel orifice, the barrel orifice defining an opening to the interior cavity and the flange configured to securedly receive the barrel cap.

3. The syringe device of claim 2, wherein the flange extends outward asymmetrically about the barrel orifice.

4. The syringe device of claim 2, wherein the flange extends outward symmetrically about the barrel orifice.

5. The syringe device of claim 2, wherein the barrel cap is removably engaged to the barrel end.

6. The syringe device of claim 3, wherein the barrel cap removably engages the flange.

7. The syringe device of claim 5, further comprising:
threading structures positioned about the barrel orifice; and
wherein the barrel cap defines threading for engaging the threading structures.

8. The syringe device of claim 7, wherein the threading structures about the barrel orifice are located above the flange.

9. The syringe device of claim 8, comprising therapeutic mammalian cells within the interior cavity, wherein the therapeutic mammalian cells are a component of the therapeutic composition.

10. The syringe device of claim 9, further comprising:
a secondary seal positioned between the barrel cap and the barrel end, and selected from a an induction seal or a gasket.

11. (canceled)

12. The syringe device of claim 1, further comprising:
a needle cap for sealing the needle end, comprising:
a syringe needle end engagement surface defining tip threaded structures for engaging needle end threading structures at the interior wall of the needle end.

13. The needle cap of claim 12, further comprising:
an elastomeric stopper for engaging a needle tip orifice defined at the needle end
a user engagement surface, wherein the needle engagement surface is spaced inwardly from the user engagement.

14. (canceled)

15. A barrel cap for sealing a syringe barrel, comprising:
an exterior surface;
an interior surface, comprising:
one or more structures for removably engaging one or more complementary structures at or near a barrel end of the syringe, the one or more structures extending outward from the syringe; and
a secondary seal configured for placement between the interior of the barrel cap and the barrel end of the syringe.

16. The barrel cap of claim 15, wherein the secondary seal is one or more of an induction seal or a gasket.

17. The barrel cap of claim 15, wherein the one or more structures include one or more of a plurality of tabs spaced about the interior surface, a first threading, cam structures, and snap taps, and engage an exterior side of the complementary structures, the complementary structures extending away from the barrel end.

18-22. (canceled)

23. A method for preventing ingress of contaminants into a syringe lumen, comprising:
placing a therapeutic composition within the syringe lumen of a syringe;
placing at least a portion of a plunger in the syringe lumen, wherein the therapeutic compound is positioned in the lumen between a needle end and the plunger; and
sealing the syringe lumen at a barrel end with a barrel cap.

24. The method of claim 23, wherein the syringe lumen is sealed at the needle end of the syringe with a needle tip cap.

25. The method of claim 24, further comprising
placing a secondary seal between the needle tip cap and the needle end or between the barrel cap and the barrel end;
induction sealing an orifice providing access to the syringe lumen; and
freezing the syringe.

26. The method of claim 25, wherein:
the secondary seal is one or more of an elastomeric stopper, a gasket, or an induction seal.

27. (canceled)

28. A method of inductively sealing a syringe, wherein induction sealing includes;
placing an inductive seal at or near a barrel end opening;
applying a compressive force to the inductive seal, in the direction of the barrel end opening; and
subjecting the inductive seal to an electromagnetic field, wherein the electromagnetic field is sufficient to heat a metal portion of the inductive seal to a temperature sufficient to melt a polymer of the inductive seal, wherein the syringe lumen includes a therapeutic mammalian cell composition.

29. (canceled)
